(19) United States
(12) Patent Application Publication
(10) Pub. No.: US 2005/0124902 A1
(43) Pub. Date: Jun. 9, 2005

(54) Electrical and audio anatomy-signal sensor system

(75) Inventors: Martin Baumer, Carlton, OR (US); Jagtar S. Saroya, Washougal, WA (US)

Correspondence Address: ROBERT D. VARITZ, P.C., 2007 S.E. GRANT STREET, PORTLAND, OR, 97214, US

(73) Assignee: Inovise Medical, Inc.

(21) Appl. No.: 10/998,704

(22) Filed: Nov. 29, 2004

Related U.S. Application Data
(60) Provisional application No. 60/526,351, filed on Dec. 1, 2003.

(51) Int. Cl. A61B 5/04; A61B 5/02; A61B 13/00

(52) U.S. Cl. 600/509; 600/528; 600/372; 600/391

(57) ABSTRACT

A sensor system for the combined, simultaneous, common-axis collection of heart-produced acoustical and electrical signals. The system includes (a) a hollow-interior body of revolution possessing an axis of revolution which is substantially coincident with the common signal-collection axis, (b) an electrode structure mounted on the outside of the body and disposed symmetrically with respect to the signal-collection axis, (c) a sound transducer disposed within the interior of the body, substantially centered on the signal-collection axis, (d) a cushion of revolution circumsurrounding and supporting the transducer within the body in a manner which substantially isolates the sound transducer from spurious noise events, and (e) an anatomy-adhereable acoustic membrane, disposed in the path for sound collection to act as a mechanical anatomy-to sensor impedance-matching coupler assisting in sound-signal collection.

### CROSS REFERENCE TO RELATED APPLICATION

This application claims priority to U.S. Provisional Patent Application Ser. No. 60/526,351, filed Dec. 1, 2003, for “Electrical and Audio Anatomy-Signal Sensor System”. All of the disclosure materials contained in that currently pending and earlier filed provisional case are hereby incorporated herein by reference.

### BACKGROUND AND SUMMARY OF THE INVENTION

This invention relates to a combined electrical and audio anatomy-signal sensor system suitable for the collection of simultaneous anatomy signals, such as ECG electrical and heart-generated acoustic, or sound, signals, from a substantially common anatomical site on the outside of the anatomy of a human subject. In particular, the invention relates to such a system in which special features are incorporated to promote very high-efficiency and accurate collection of such signals.

A preferred and best-mode embodiment of, and manner of practicing, the present invention are illustrated and described herein in the specific area of gathering heart-produced signals with respect to which the invention has been found to offer particular utility. It should be understood, however, that specific reference to the term “heart” herein is intended to refer illustratively, and as a representative surrogate, also to and for various “other-source” anatomy signals.

An so, progressing now with a description of the background for the present invention, with reference particularly in the setting of the heart, there are many medical-related reasons why it is considered important today, in the practice of heart-related medicine, to collect a variety of ECG electrical, as well as acoustic, heart-generated signals, for the purpose of examining these signals as a way of discerning, diagnosing and treating a subject's current heart condition. In order for this practice to be successful and reliable, it is very important that collected signals be gathered in such way that they clearly distinguish over background noise and other extraneous signal sources, with extraneous sound sources often presenting the “lion's share” of culprit signal behavior in this practice. The present invention addresses this area of medical technology by offering an extremely compact, simple, and relatively inexpensive combinational sensor system which allows for high-reliability gathering, simultaneously, of ECG electrical and heart-generated acoustical signals effectively from a shared, or common, anatomical site, utilizing a unique electrode structure for collecting ECG electrical signals, and a uniquely supported microphone (sound transducer) for collecting acoustical signals, both together effectively acquiring these signals along what is referred to herein as a single common axis for signal collection.

As will be seen, the sensor system of the present invention features a unique arrangement wherein, within the hollow interior of a unitary sensor body of revolution, a microphone (sound transducer) is effectively floatingly shock-mounted within an elastomeric boot, also referred to as a cushion of revolution herein, which functions extremely effectively directly to support and isolate the microphone from extraneous mechanical noise. This same boot also definitively assists in sealing a sound-reception air space (acoustic chamber) formed within the sensor body, and dedicated to the microphone for the purpose of collecting acoustic signals. This air space is principally defined in the form of a domed, nominally “open-mouthed” cavity which is designed to face the anatomy during signal collection, with the open “mouth”, or side, of this cavity bridged and spanned by a novel acoustic membrane which specially assists with sound collection. The spanning membrane has an outer, generally flat surface which is intended to contact a subject's anatomy, which surface is treated with an appropriate pressure-sensitive adhesive to bond the membrane releasably to a subject's anatomy during signal collection, whereby the membrane acts extremely effectively as an acoustic impedance-matching coupling component in the sound path existing between the floating microphone and a subject's anatomy. A small, centralized aperture in the membrane, centered on the mentioned common axis for signal collection, allows for appropriate pressure equalization on opposite sides of the membrane prior to attachment of the sensor system to a subject's anatomy.

Cooperating with this acoustical structure is an electrode structure which takes the form of a broad skirt-like expanse of a thin, carbon-vinyl laminate, electrical-signal-gathering instrumentality which circumsurrounds and radiates outwardly from the body of the sensor which contains the microphone. The outwardly radiating portion of this skirt-like expanse includes, centrally, what is referred to herein as an annular curtain ring of “star-burst” fingers which extend up along the outer side walls of the sensor body to provide, during use, a robust electrode possessing an electrical signal conduction path between a subject's anatomy and other conductor structure which is employed to communicate all gathered sensor-electrical signals to external circuitry which is designed to receive and process, etc., these signals.

Further featured in the sensor system of this invention, and specifically within certain electrical conductor structure which is employed within the sensor system sensor body to communicate electrical signals between the sensor and the “outside world”, is a passive electrical component, preferably in the form of a particular selected-value resistor which can be employed by connected external circuitry to identify very clearly the nature and character of the sensor system which is supplying signals. This is an important “branding” consideration where someone using the system of this invention needs to know that, in fact, the signals which are being collected have the expected reliability which characterizes the operation of this invention. In other words, an incorrect sensor device connected for feeding signals would not be identified as a correct sensor device, and its identity would not be reported to the external circuitry as being the identity of the desired sensor device—namely, the sensor system of this invention.

These and various other features and advantages which are attained by the invention will become more clearly apparent as the description which shortly follows is read in conjunction with the accompanying drawings.

### DESCRIPTION OF THE DRAWINGS

FIG. 1 is an isometric, modestly detailed and partially fragmentary, view of a fully integrated and compact anatomical-signal sensor system constructed in accordance with the present invention adhered to the anatomy (also shown fragmentarily) for collecting signals.

FIG. 2, which is presented on a somewhat larger scale than that employed in FIG. 1 illustrates a side view of the sensor system shown in FIG. 1.

FIG. 3, drawn on an even larger scale than that used in FIG. 2, illustrates a fragmentary, central cross section through the sensor system of FIGS. 1 and 2, taken in a plane which transects the sensor system and which contains what is referred to herein as the axis of revolution of certain components in the system.

FIGS. 4 and 5 are very similar to one another, with each illustrating an exploded view of the principal components which make up the sensor system of FIGS. 1-3, inclusive.

FIG. 6 is an isometric and isolated computer-rendered top view showing a sensor body employed in the system of the invention.

FIGS. 7 and 8 are, respectively, top-isometric and bottom-isometric, computer-rendered views of an elastomeric boot, or cushion of revolution, which is employed according to the invention to support a microphone within the body pictured in FIG. 6.

FIG. 9 is a bottom, computer-rendered view essentially taken along the axis of revolution of the system of this invention, and clearly illustrating both a centrally apertured acoustic membrane which is employed in the system of the invention, and a circumsurrounding layer of conventional, electrically conductive, anatomy-adhering hydrogel.

FIG. 10 is an isometric view illustrating a nearly completely assembled sensor system, clearly showing a portion of what is referred to herein as an annular curtain-ring of conductive, thin, carbon “star-burst” fingers which extend upwardly along and around a portion of the outside of the body-of-revolution sensor body pictured as an isolated structure in FIG. 6. These fingers form part of a skirt-like electrode structure which is employed in the invention to gather electrical signals.

FIG. 11 is a very simplified block/schematic diagram illustrating basic electrical signal-flow connections which exist between the sensor system of this invention and external circuitry. In particular, FIG. 11 illustrates the earlier-mentioned, selected-value, passive electrical resistor component which is employed in the system of this invention to provide a clear identity regarding the nature and character of the sensor system connected to the external circuitry. We refer to this component as a “branding” component.

### DETAILED DESCRIPTION OF THE DRAWINGS

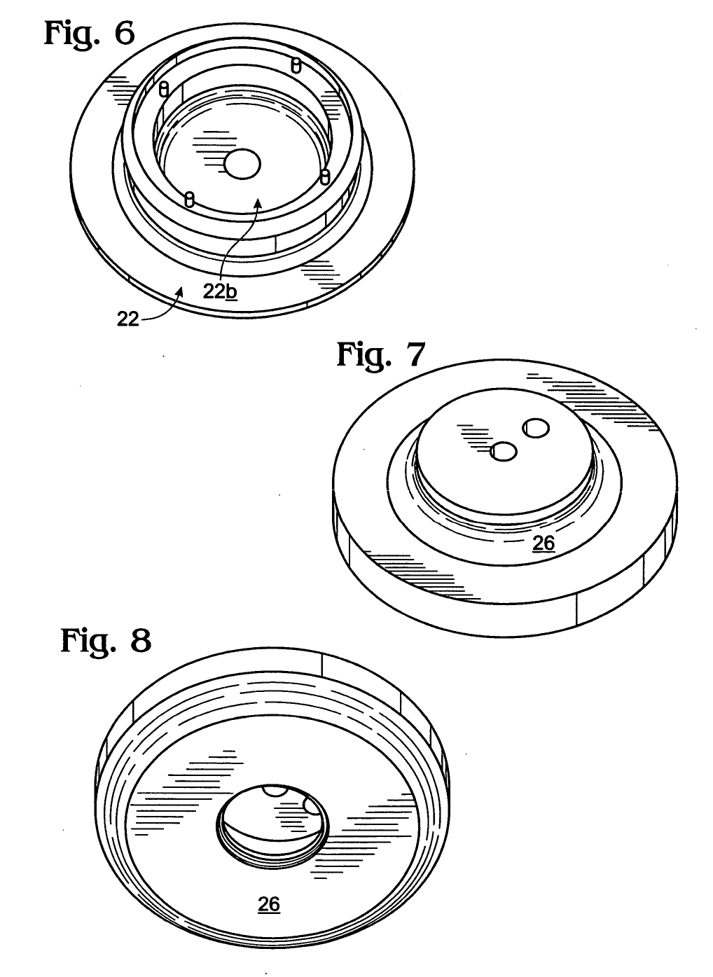

Turning attention now to the drawings, and referring first of all to FIGS. 1-10, inclusive, indicated generally at 20 is an anatomical-signal-sensor system constructed in accordance with a preferred and best-mode embodiment of the invention. System 20 generally includes (a) a unitary sensor body of revolution 22 possessing an axis of revolution 22a, (b) a microphone, or sound transducer, 24 which is centered on axis 22a and shock-mounted and isolated within the hollow interior 22b in body 22 by (c) an elastomeric boot, or cushion of revolution (or vibration-isolating resiliency component), 26, (d) a generally skirt-like laminated vinyl and carbon electrode, or electrode structure, 28, and (e) a thin, centrally apertured (through-passaged) acoustic membrane 30 which spans the open mouth 22c of a domed-surface (22d) acoustic, or sound-collection, chamber 22e. The aperture (or through-passage) in membrane 30 is shown at 30a.

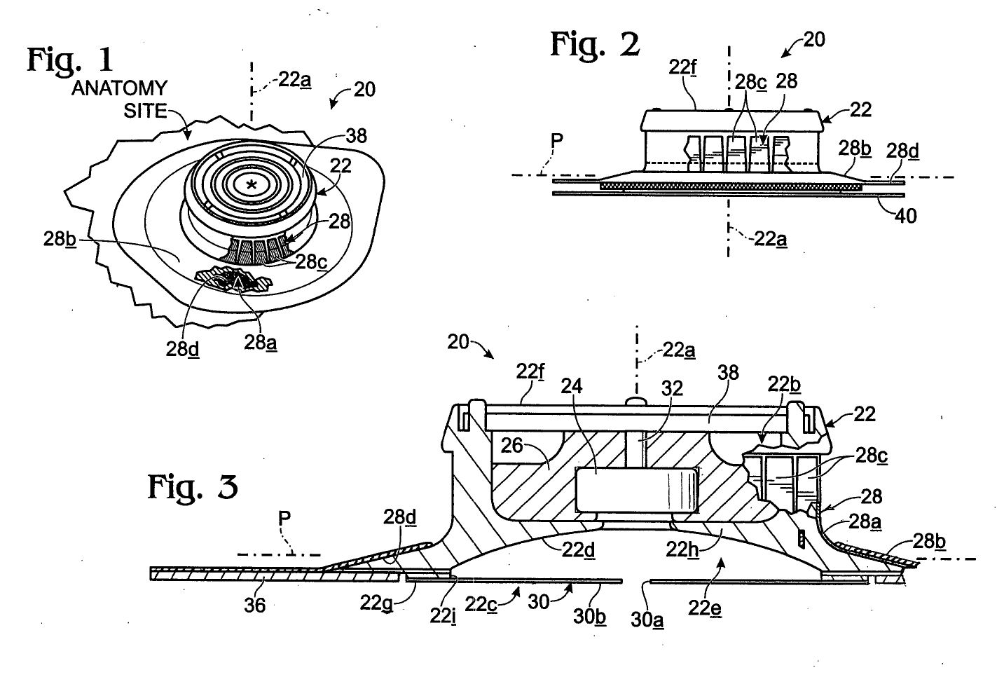

Body 22 preferably is formed from a suitable, molded, non-electrically conductive, ABS plastic material to have the configuration and axial cross-section which are clearly shown in FIGS. 2, 3-6, inclusive, and 10. The upper, relatively cylindrical portion of body 22, which portion possesses the upper face 20, or side, 22f in the body, has an outside diameter of about 0.75-inches, and an axial height of about 0.625-inches. The lower, outwardly flared portion of body 22, clearly illustrated in the drawings, which portion possesses the axially opposite, lower face, or side, 22g in the body, has an outside diameter of about 1.25-inches, and axial height of about 0.125-inches. Nominally, the hollow interior 22b in body 22, which is somewhat axially divided into two clearly evident (see especially FIG. 3), axially spaced regions by an open-centered annular wall 22h, opens to opposite faces, or sides 22f, 22g in the body. The lower side of wall 22h in the sensor body, as such is especially well seen in FIG. 3, is the previously mentioned domed surface 22d in the sensor body. Surface 22d herein is generally spherical in configuration, is substantially symmetrically centered on axis 22a, and joins face 22g through a substantially cylindrical, axially relatively short wall portion 22i.

Boot is preferably formed of a suitable rubber vibration-damping elastomer, has the axial cross-sectional configuration pictured in FIG. 3, and snuggly and circumsurroundingly supports microphone 24 in the position shown in FIG. 3—centered on axis 22a, and aimed into chamber 22e toward sensor body mouth 22c. As can be seen, boot 26 occupies the upper, otherwise unfilled portion of body interior 22b, and both stabilizes microphone 24 within body 22, and floatingly isolates it from spurious vibrations transmitted from the outside into the sensor body. Boot 26 also conveniently functions to furnish an acoustical seal between microphone 24 and sensor body 22.

Appropriate electrical conductors 32 extend from microphone 24 axially through boot 26 toward and through the upper side of the boot in FIG. 3, which upper side faces the upper region of body interior 22b.

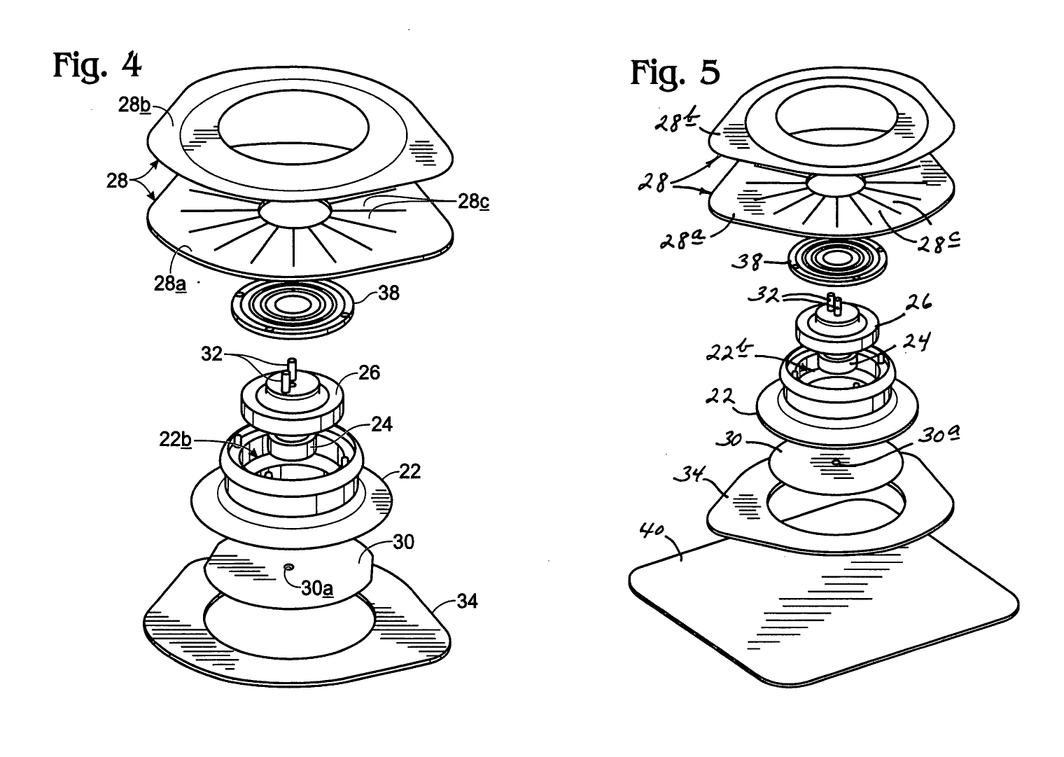

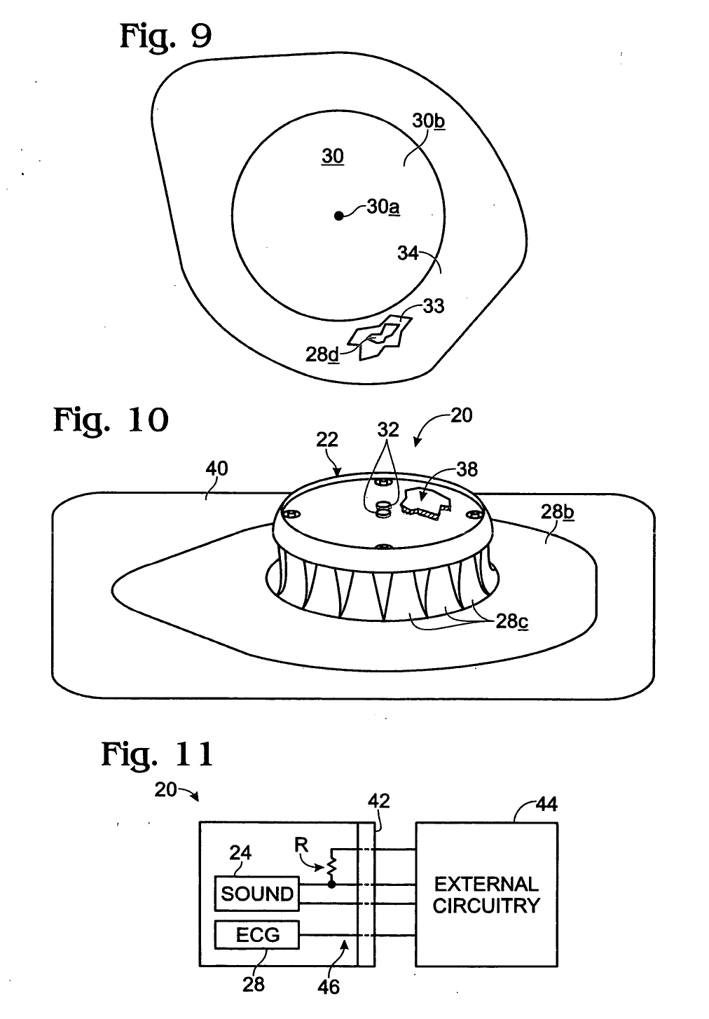

Electrode structure 28 which is preferably a laminate structure, or laminate, is herein formed of a suitable thin sheet of carbon 28a which has been suitably bonded to the underside of a thin vinyl carrier 28b, nominally, i.e., before assembly into system 20, has the undeformed configuration illustrated for it clearly in FIGS. 4 and 5. It can plainly be seen in these two figures that this electrode structure, and in particular carbon sheet 28a, possesses what can be thought of as a central, star-burst pattern cut into it. During assembly of system 20, this laminate electrode structure is pressed downwardly over body 22, from the cylindrical end thereof, thus causing to occur the obvious central deformation of the cut star-burst pattern in the carbon sheet portion of the electrode laminate, thereby resulting in the final electrode configuration seen especially well in FIGS. 2, 3 and 10. This resulting configuration can be characterized as being flared and skirt-like, with a central, “upstanding”, annular curtain-ring of the fingers 28c which ride against the cylindrical outside wall of the upper, cylindrical portion of body 22, and a generally radially outwardly annular and somewhat planar (slightly inclined) skirt portion 28d. The nominal plane of this skirt portion of the electrode structure is shown generally at P in FIGS. 2.and 3. The sides of fingers 28c which ride against the sensor body are appropriately coated with a suitable pressure-sensitive adhesive whereby they bond to the sensor body. The underside of skirt portion 28d is suitably coated with an electrically conductive silver-silver fluoride coating 33 (see particularly FIG. 9).

Electrode structure 28, together with a conventional underside-adhered layer 34 of hydrogel, functions to collect anatomical electrical signals, such as heart-produced ECG signals, symmetrically with respect to sensor body access 22a.

From what has been described so far herein in this detailed description of the invention, it will be seen that microphone 24, and electrode structure 28 (assisted by hyrdogel layer 34), collect acoustical and electrical anatomy signals, respectfully, substantially symmetrically with respect to axis 22a. Axis 22a is also referred to herein as a common signal-collection axis.

Acoustical membrane 30 takes the form herein of a thin (about 0.002-inches) circular expanse of polyethylene, with previously mentioned central aperture, or through-passage, 30a herein having a diameter of about 0.040-inclues. This membrane is firmly bonded to the circular rim of mouth 22c at the base of chamber 22e, as can clearly be seen in FIG. 3. Its lower, outwardly-facing, “anatomy-contacting surface 30b is coated with a thin layer of suitable pressure-responsive anatomical medical adhesive 36, which may be any well known suitable and conventional anatomical adhesive material. This membrane underside adhesive layer, during use of sensor system 20, bonds with pressure sensitivity to a selected site in the anatomy to cause the membrane to function as a relatively high-efficiency acoustical matching (impedance matching) structure for the accurate and excellent delivery of sound signals to microphone 24 through chamber 22e. Preferably the adhesives chosen for use with respect to the membrane are such that the adhesive employed between the membrane and sensor body 22 produces an appreciably higher tenacity of bonding therebetween than the bonding tenacity which develops with a subject's anatomy through the adhesive which exists on the underside of the membrane. The central aperture 30a in membrane 30 functions, before attachment of system 20 to the body, to assure pressure equalization on opposite sides of the membrane—a feature which contributes to ultimate accuracy of sound-information collection.

Completing a description of the various components of the invention as pictured in FIGS. 1-10, inclusive, shown at 38 in FIG. 1 is a thin, circular, printed circuit board which includes bulls-eye-like circular traces 40 of conductors which connect, on the underside of this circuit-board component, with previously mentioned conductors 32. An easy-release liner formed of polyethylene, and shown at 40, is initially attached adhesively to the underside of a fully completed sensor system 20 to protect the body-contacting surface area(s) before system 20 is put into use. When the sensor system is to be used, this releasable liner is removed to expose the operative underside of sensor system 20 for attachment to the anatomy.

Including attention now to FIG. 11 in the drawings, forming no part of the present invention, but employed to receive electrical ECG signals collected by electrode structure 28, and sound-to-electrical-converted signals collected (and converted) by microphone 24, an adapter (shown only schematically herein at 42 in FIG. 11), somewhat like the adapter described in currently co-pending, prior-filed U.S. patent application Ser. No. 10/426,098, filed Apr. 29, 2003, for “Electrical and Audio Anatomy-Signal Sensor/Coupler-Adapter Interface”, is removeably snap coupled to the upper, cylindrical portion of sensor body 22. Such an adapter is appropriately equipped to make electrical contact with the conductive traces formed on circuit board 38, and also to make contact with the upper ends of fingers 28c on the outside of sensor body 22.

In FIG. 11, an operative situation is schematically illustrated, with sensor system 20 here shown connected through adapter 42 to external circuitry which is represented by a block 44. Included within system 20, herein on the underside of printed circuit board 38, is a passive electrical component in the form of a resistor which is labeled R. This resistor is connected to the sound-signal portion of appropriate electrical signal conductor structure 46 which is included within system 20 to convey electrical signals “outwardly” from the system. Resistor has what is referred to herein as a selected value, and functions to provide a capability at the site of external circuitry to identify sensor system 20 as an appropriate sensor system to be employing with respect to appropriate reception circuitry included in the external structure. We refer to this component, resistor R, as a branding component.

The sensor system of this invention is now fully described and illustrated in its preferred and best-mode forms. The proposed system is simple and compact, and relatively easy and inexpensive to construct. It accommodates reliable and robust electrical and sound signal collection from a single anatomical site along a single signal-collection axis. Special floating and sound-isolation mounting is provided for the included sound-signal transducer, which is furnished sound signals through a domed and sealed chamber via a special signal-coupling, impedance-matching membrane which becomes bonded to the selected anatomical site on the anatomy of a subject. A unique skirt-like electrode structure cooperates symmetrically with the sound transducer to acquire anatomy-produced electrical signals along the same signal-collection axis associated with the sound transducer.

Accordingly, while specific disclosure of the invention has been provided herein, it is appreciated that variations and modifications may be made in this subject matter well within the scope of the invention.

1. A sensor system for the combined, simultaneous, common-axis collection of heart-produced acoustical and electrical signals comprising
a body of revolution possessing an axis of revolution which is substantially coincident with said common axis, and having a hollow interior, and a pair of opposite faces spaced along, and substantially centered on, said axis of revolution and opening to said interior,
an electrical electrode structure mounted on the outside of said body and disposed angularly symmetrically with respect to said axis of revolution,
a sound transducer disposed within said interior, substantially centered on said axis of revolution, and oriented to collect acoustical signals entering said body through one of said faces, and
a cushion of revolution circumsurrounding and supporting said transducer in a manner which substantially fills the majority of said hollow interior.

2. The sensor system of claim 1, wherein, within an unfilled portion of said hollow interior, intermediate said transducer and said one face, there is formed a sound-collection chamber having a domed surface substantially centered on said axis of revolution and facing outwardly toward said one face.

3. The sensor system of claim 1 which further includes an acoustic membrane spaced from said transducer and substantially spanning said one face, and disposed for operative engagement with a subject's anatomy.

4. The sensor system of claim 3, wherein said membrane possesses a central aperture substantially centered on said axis of revolution.

5. The sensor system of claim 3, wherein said membrane has an anatomy-contacting surface, and which further comprises an anatomy-adhering, pressure-sensitive adhesive layer distributed over said surface.

6. The sensor system of claim 2 which further includes an acoustic membrane spaced from said transducer and substantially spanning said one face, and disposed for operative engagement with a subject's anatomy.

7. The sensor system of claim 6 wherein said membrane possesses a central aperture substantially centered on said axis of revolution.

8. The sensor system of claim 6, wherein said membrane has an anatomy-contacting surface, and which further comprises an anatomy-adhering, pressure-responsive adhesive surface layer distributed over said surface.

9. The sensor of claim 1, wherein said electrode includes an annular curtain-ring of fingers distributed in contact with, and in a fashion circumsurrounding, said body of revolution, said fingers joining with an outwardly flared, generally annular and planar skirt portion circumsurrounding said body generally in a plane which is normal to said axis of revolution in a region adjacent said body's said one face.

10. The sensor system of claim 1 which further comprises electrical signal conductor structure including respective portions operatively connected to said transducer and to said electrode for accommodating signal coupling to selected external circuitry, said conductor structure possessing an electrical component which, with respect to an established connection with such external circuitry, effects an identification which is unique to said system.

11. The sensor system of claim 10, wherein said electrical component is associated with that portion of said conductor structure which connects with said transducer.

12. The sensor structure of claim 10, wherein said electrical component comprises a selected-value resistor.

13. A sensor system for collecting anatomy-produced signals comprising
a sensor body including an internal acoustic chamber with a mouth exposed to one side of said body adapted to face a subject's anatomy for the purpose of receiving anatomy-produced acoustical signals,
a sound transducer mounted within said body and operatively and communicatively exposed to said chamber,
an acoustic membrane spanning said mouth, and having one side facing said chamber and an opposite side facing away from the chamber, and
an anatomy adhesive layer distributed over said membrane's said opposite side accommodating releasable bonding of the membrane's said opposite side to a selected site on a subject's anatomy.

14. The sensor system of claim 13, wherein said membrane includes a pressure-equalizing through-passage which opens to the one and opposite sides of the membrane.

15. The sensor system of claim 13 which further includes a vibration-isolating resiliency component supporting said transducer within said sensor body at a location spaced from said mouth and membrane.
